(19) United States
(12) Patent Application Publication
(10) Pub. No.: US 2009/0047310 A1
(43) Pub. Date: Feb. 19, 2009

(54) Use of a griffonia extract, in particular griffonia simplicifolia, in a cosmetic or dermatological composition for mitigating pigmentation of skin and skin appendages

(76) Inventor: Alain Meybeck, Courbevoie (FR)

Correspondence Address: JACOBSON HOLMAN PLLC, 400 SEVENTH STREET N.W., SUITE 600, WASHINGTON, DC, 20004, US

(21) Appl. No.: 11/921,943

(22) PCT Filed: May 24, 2006
(86) PCT No.: PCT/FR2006/001181

§ 371 Date: Mar. 10, 2008

(30) Foreign Application Priority Data
Jun. 13, 2005 (FR) 0505958

(51) Int. Cl. A61K 8/97; A61K 8/49; A61Q 19/02; A61Q 17/04

(52) U.S. Cl. 424/401; 424/769; 514/419; 424/62; 424/59; 424/744; 424/741

(57) ABSTRACT

Use of an extract of Griffonia, in particular of Griffonia simplicifolia, in a cosmetic or dermatological preparation for attenuating the pigmentation of the skin and of superficial body growths.

This cosmetic or dermatological preparation may also contain any of the following products: at least one other active depigmenting product, at least one substance for filtering or blocking B and/or A ultraviolet radiation, at least one anti-inflammatory product, at least one agent encouraging flaking. It contains from 0.05 to 20% by weight of an extract of Griffonia simplicifolia.

The present invention relates to the use of an extract of Griffonia, in particular of Griffonia simplicifolia, in a cosmetic or dermatological preparation for attenuating the pigmentation of the skin and of superficial body growths.

Griffonia simplicifolia is a tree growing mainly in West Africa, and more particularly in Ghana, Ivory Coast and Togo. Its stems are used locally to relieve intercostal pain and its leaves to treat liver disease and to suppress vomiting (Plantes Médicinales d'Afrique, J-L Pousset, Edisud, 2004). The seeds of Griffonia simplicifolia are currently the only source for the industrial production of 5-hydroxytryptophan (5-HTP). This molecule is used in pharmacy as an anti-depressant and an anti-epileptic and in the field of food additives as a precursor of serotonin for treatment of headaches, appetite and sleep disorders, as described in articles published in Nutranews, July 2004 and in Phytochem. Anal. 13, 333-337 (2002).

This type of use is described in particular in the documents US-2004/0197377-A, US-2003/0077233-A and EP-1502597-A.

The coloration of the skin and of superficial body growths (hair, nails, etc.) is due to brown pigments secreted by specialized cells known as melanocytes by a complex biochemical process involving more than 90 genes (Tadokoro T., in Mechanisms of Suntanning, published by J-P Ortonne and R Ballotti, Martin Dunitz, 2002, p. 67).

In many cultures, and in Asia in particular, it is preferred to have the whitest possible skin, or at least no pigmentation marks.

This is why the cosmetic industry markets a large number of products intended to prevent the skin from tanning, to whiten the skin or to attenuate marks on the skin. There are also dermatological preparations intended to attenuate pigmentation marks such as “stretch marks” or to whiten the skin around the white spots of vitiligo so that they are less visible.

Thus substances are known that are used to attenuate cutaneous pigmentation, such as: hydroquinone and its derivatives, such as arbutin, kojic acid, azelaic acid, octadecenedioic acid, certain plant extracts such as extracts of Morus alba and Glycyrrhiza glabra, ascorbic acid and its derivatives such as ascorbyl phosphate or the double phosphate of vitamin C and vitamin E, certain derivatives of cysteine, ecdysterone, etc.

The document FR-2.848.846-A describes one example of an agent for depigmentation and/or whitening of the skin and/or superficial body growths in the form of derivatives of ascorbic acid.

The document FR-2.841.550-A describes another example consisting of molecules derived from tyramine.

However, the above substances are sometimes cytotoxic to melanocytes and/or insufficiently effective and/or difficult to use; for example, preparations based on hydroquinone are currently not freely on sale.

Thus one aim of the present invention relates to the use of a cosmetic or dermatological preparation whose active ingredient does not have the drawbacks explained above.

A further aim of the invention is to provide a preparation of the above kind of improved efficacy at competitive cost.

The above aims, and others that will become apparent hereinafter, are achieved by the use of an extract of Griffonia in a cosmetic or dermatological preparation for attenuating the pigmentation of the skin and of superficial body growths.

The extract of Griffonia is preferably an extract of Griffonia simplicifolia and to be more precise an extract of the seeds thereof.

The extract of Griffonia is advantageously an aqueous, hydro-alcohol, hydro-glycol or hydro-glycerol extract containing 5-hydroxytryptophan.

This preparation preferably contains from 0.05 to 20%, and to be more precise from 0.5 to 5%, by weight of an extract of Griffonia simplicifolia.

The extract of Griffonia advantageously contains from 0.1 to 100%, and preferably at least 3%, by weight of 5-hydroxytryptophan.

This cosmetic or dermatological preparation containing an extract of Griffonia for attenuating the pigmentation of the skin and of superficial body growths, as above, may also contain any of the following products:
at least one other active depigmentation product such as arbutin, kojic acid, azelaic acid, octadecenedioic acid, extract of Morus alba, extract of Glycyrrhiza glabra, ascorbic acid and its derivatives such as ascorbyl glucoside, ascorbyl phosphate and the double phosphate of vitamin C and vitamin E, a derivative of cysteine, ecdysterone, ellagic acid, etc.,
at least one substance for filtering or blocking B and/or A ultraviolet radiation such as pigments based on titanium oxide, nacreous pigments, molecules of type benzophenone, anthranilate, cinnamic, dibenzoyl methane, salicylic, derivatives of camphor, etc.,
at least one anti-inflammatory product such as extract of Glycyrrhiza glabra, glycyrrhetinic acid, extract of Scutellaria baicalensis, extract of Aloe vera, bisabolol, etc.,
at least one agent encouraging flaking such as salicylic acid, alpha-hydroxy acids, urea, etc.,

The extract of Griffonia is advantageously incorporated into particles such as liposomes, vesicles, dispersed lamellar phases, capsules, sponges, of size from 1 nm to 100 microns, and to be more precise from 10 nm to 10 microns.

The extract of Griffonia is preferably a powder.

This cosmetic or dermatological preparation containing an extract of Griffonia for attenuating the pigmentation of the skin and of superficial body growths may advantageously be in one of the following forms:
an aqueous or hydro-alcohol lotion, an aqueous or hydro-alcohol gel, a micro-emulsion, a liquid or thick emulsion, a loose or compacted powder,
a patch, the preparation being disposed on a solid support,
an ingestible form.

This cosmetic or dermatological preparation containing an extract of Griffonia for attenuating the pigmentation of the skin and of superficial body growths is advantageously in the form of a capsule or tablet containing from 5 mg to 200 mg of 5-hydroxytryptophan, and preferably from 25 to 75 mg of 5-hydroxytryptophan.

As indicated above, and entirely surprisingly, it has been found that extracts of the seeds of Griffonia simplicifolia have a significant action in terms of attenuating the pigmentation of the skin and of superficial body growths, and there is no doubt that this is achieved by operating on the production of melaninic pigments by the melanocytes.

The following non-limiting description relates in particular to embodiments of the present invention that will enable the person skilled in the art to understand the present invention better.

### EXAMPLE 1

### Obtaining Extracts of the Seeds of Griffonia simplicifolia

An extract of seeds of Griffonia simplicifolia is prepared by a method known in the art using methanol and water.

From 500 g of seeds 110 g of a dry extract are obtained using 4 liters of a 50% hydromethanol mixture.

2.5 g of this dry extract are then dissolved in 250 ml of cold water and extracted twice with 100 ml of n-butanol:
the organic phase is evaporated to obtain a butanol fraction;
the aqueous phase is dried in a rotary evaporator and the dry residue taken up in 12 ml of boiling water to obtain 270 mg of a recrystallized aqueous fraction after filtration and cooling.

### EXAMPLE 2

### Chromatographic Comparison with 5-HTP

The dry extract, the butanol fraction and the recrystallized aqueous fraction obtained in example 1 are submitted in the presence of a 5-hydroxytryptophan (5-HTP) control to thin film chromatography using as the eluant an acetone/chloroform/acetic acid/water mixture in proportions by volume of 8:8:4:1 and a solution of ninhydrin in butanol as the revealer.

This analysis shows that 5-HTP is present in the dry extract, the butanol fraction and the recrystallized aqueous fraction.

### EXAMPLE 3

### Study of the Activity of the Extracts on Melanogenesis

The action on the biosynthesis of melanin of the three products extracted from Griffonia simplicifolia obtained in example 1 is studied.

a)—Effect of the Dry Extract

Normal human melanocytes are cultured for ten days in the absence and in the presence of non-cytotoxic quantities of the dry extract. The quantity of melaninic pigments is evaluated by extraction using 0.5 N soda and optical density measurement.

The dry extract revealed significant melanogenesis inhibiting activity with a p less than 0.01 at the concentration of 0.004 mg/ml.

b)—Effect of the Butanol Fraction

The procedure is as before for the dry extract.

The butanol fraction at the concentration of 0.04 mg/ml led to a significant reduction in the quantity of melanin produced by the melanocytes; only 86% is found of the quantity obtained in the control culture (p less than 0.01).

c)—Effect of the Recrystallized Aqueous Fraction

The procedure is as before for the dry extract.

The recrystallized fraction at the concentrations of 0.04 mg/ml, 0.008 mg/ml and 0.0016 mg/ml led to a significant and dose-dependent reduction in the quantity of melanin produced by the melanocytes: respectively 84 and 89% of the control (p less than 0.01) and 92% of the control (p less than 0.05).

### EXAMPLE 4

### Effects of 5-HTP on Melanogenesis

The effect of 5-HTP on the production of melanin by the melanocytes is studied.

The procedure is as before for the dry extract using 5-HTP extracted from the seeds of Griffonia simplicifolia.

The 5-HTP at the concentrations of 0.04 mg/ml, 0.008 mg/ml and 0.0016 mg/ml reduced significantly and in a dose-dependent manner the quantity of melanin produced by the melanocytes: respectively 80 and 89% of the control (p less than 0.01) and 93% of the control (p less than 0.05).

The above examples show that the extracts of Griffonia simplicifolia contain 5-hydroxytryptophan (5-HTP) and have a significant action on the production of melanin just like 5-HTP itself.

Consequently, cosmetic or dermatological preparations can be produced with the aim of attenuating the pigmentation of the skin and of superficial body growths. A few nonlimiting examples of such preparations are given hereinafter:

Preparation No 1: Cosmetic Cream

The procedure is as for the production of a conventional oil in water emulsion, i.e. there are mixed under agitation at 85° C. a hydrophobic phase and an aqueous phase each containing raw materials intended to confer the usual properties of use on this kind of preparation (stability, safety, easy spreading, etc.).

Cooling under agitation to a temperature below 45° C. is followed by the addition of 1% by weight of a purified extract of Griffonia simplicifolia prepared like the above recrystallized fraction.

Preparation No 2: Preventive Cosmetic Product

A cosmetic product for preventing cutaneous pigmentation is produced in accordance with the following formula:

| | |
|---|---|
| Dry hydromethanol extract of Griffonia simplicifolia | 1 |
| (dry extract of example 1) | |
| Oil in water emulsion excipient containing | qsp 100 |
| ultraviolet filters | |

The procedure for producing this preparation No 2 is the same as for preparation No 1.

Preparation No 3: Cosmetic Gel

A cosmetic gel for whitening the skin is prepared in accordance with the formula:

| | |
|---|---|
| Recrystallized fraction of Griffonia simplicifolia | 0.5 |
| of example 1 | |
| Proliposome phospholipids | 2 |
| Water | 50 |
| Aqueous gel | qsp 100 |

The recrystallized fraction is dissolved in water; the resulting solution is then homogenized and the aqueous gel added under moderate agitation.

Preparation No 4: Dermatological Formulation

A dermatological formulation for whitening the skin is prepared in accordance with the formula:

| | |
|---|---|
| 5-hydroxytryptophan (5-HTP) extracted from | 3 |
| Griffonia simplicifolia | |
| Gelified hydro-alcohol excipient | qsp 100 |

The 5-HTP is dissolved in the excipient to obtain this kind of preparation.

Preparation No 5: Cosmetic Cream

A whitening cosmetic cream combining an extract of Griffonia simplicifolia with other active ingredients for attenuating cutaneous pigmentation is prepared, as for preparation No 1, in accordance with the following formula:

| | |
|---|---|
| 5-hydroxytryptophan (5-HTP) extracted from | 3 |
| Griffonia simplicifolia | |
| Ecdysterone | 0.2 |
| Arbutin | 1 |
| Oil in water emulsion excipient including | qsp 100 |
| ultraviolet filters | |

Preparation No 6: Capsules to be Ingested

Capsules containing 50 mg of 5-HTP are prepared: the recommended daily dose is one capsule, preferably taken 30 minutes before the last meal of the day.

Obviously, a preparation according to the present invention may also contain any of the following products:
at least one other active depigmenting product such as arbutin, kojic acid, azelaic acid, octadecenedioic acid, extract of Morus alba, extract of Glycyrrhiza glabra, ascorbic acid and its derivatives such as ascorbyl glucoside, ascorbyl phosphate and the double phosphate of vitamin C and vitamin E, ecdysterone, a derivative of cysteine, ellagic acid,
at least one substance for filtering or blocking B and/or A ultraviolet radiation such as pigments based on titanium oxide, nacreous pigments, molecules of type benzophenone, anthranilate, cinnamic, dibenzoyl methane, salicylic, derivatives of camphor, and any other product from appendix VII of the modified EEC directive No 76/7688 on cosmetics,
at least one anti-inflammatory product such as extract of Glycyrrhiza glabra, glycyrrhetinic acid, extract of Scutellaria baicalensis, extract of Aloe vera, bisabolol,
at least one agent encouraging flaking, such as salicylic acid, alpha-hydroxy acids, urea.

This preparation may take any of the following forms:
an aqueous or hydro-alcohol lotion, aqueous or hydro-alcohol gel, micro-emulsion, liquid or thick emulsion, loose or compacted powder,
a patch, the preparation being disposed on a solid support,
an ingestible form.

Moreover, in a preparation according to the present invention, the extract of Griffonia simplicifolia or the 5-HTP may be incorporated into particles such as liposomes (as described for preparation No 3), vesicles of non-ionic amphiphilic molecules, dispersed lamellar phases, capsules and sponges, or disposed on the surface of particles such as synthetic polymer particles, cellulose particles or mineral particles.

As previously stated, the seed of Griffonia simplicifolia is at present the only industrial source of 5-HTP, which is the active ingredient in the various cosmetic or dermatological preparations according to the present invention and the use of which has the aim of attenuating the pigmentation of the skin and of superficial body growths. 5-HTP from other industrial sources, for example produced by synthesis, semisynthesis, enzyme or biotechnology processes, could be used in accordance with the present invention without departing from the scope thereof.

1. Use of an extract of Griffonia in a cosmetic or dermatological preparation for attenuating the pigmentation of the skin and of superficial body growths.

2. Use according to claim 1 of an extract of Griffonia in a preparation, characterized in that said extract is an extract of Griffonia simplicifolia.

3. Use according to claim 2 of an extract of Griffonia in a preparation, characterized in that the extract of Griffonia simplicifolia is an extract of the seeds thereof.

4. Use according to claim 2 of an extract of Griffonia in a preparation, characterized in that said extract is an aqueous extract containing 5-hydroxytryptophan.

5. Use according to claim 2 of an extract of Griffonia in a preparation, characterized in that said extract is a hydro-alcohol extract containing 5-hydroxytryptophan.

6. Use according to claim 2 of an extract of Griffonia in a preparation, characterized in that said extract is a hydro-glycol extract containing 5-hydroxytryptophan.

7. Use according to claim 4 of an extract of Griffonia in a preparation, characterized in that said preparation contains from 0.05 to 20% by weight of an extract of Griffonia simplicifolia.

8. Use according to claim 7 of an extract of Griffonia in a preparation, characterized in that said preparation contains from 0.5 to 5% by weight of an extract of Griffonia simplicifolia.

9. Use according to claim 1 of an extract of Griffonia in a preparation, characterized in that said extract contains from 0.1 to 100% by weight of 5-hydroxytryptophan.

10. Use according to claim 9 of an extract of Griffonia in a preparation, characterized in that said extract contains at least 3% by weight of 5-hydroxytryptophan.

11. Use according to claim 1 of an extract of Griffonia in a cosmetic or dermatological preparation for attenuating the pigmentation of the skin and of superficial body growths, characterized in that said preparation also contains at least one other active depigmentation product.

12. Use according to claim 11, characterized in that the other active depigmenting product is chosen from the following products: arbutin, kojic acid, azelaic acid, octadecenedioic acid, extract of Morus alba, extract of Glycyrrhiza glabra, ascorbic acid and its derivatives such as ascorbyl glucoside, ascorbyl phosphate and the double phosphate of vitamin C and vitamin E, a derivative of cysteine, ecdysterone, ellagic acid.

13. Use according to claim 1 of an extract of Griffonia in a cosmetic or dermatological preparation for attenuating the pigmentation of the skin and of superficial body growths, characterized in that said preparation contains at least one substance for filtering or blocking B and/or A ultraviolet radiation.

14. Use according to claim 13, characterized in that the substance for filtering or blocking B and/or A ultraviolet radiation is chosen from the following products: pigments based on titanium oxide, nacreous pigments, molecules of type benzophenone, anthranilate, cinnamic, dibenzoyl methane, salicylic, derivatives of camphor.

15. Use according to claim 1 of an extract of Griffonia in a cosmetic dermatological preparation for attenuating the pigmentation of the skin and of superficial body growths, characterized in that said preparations contains at least one anti-inflammatory product.

16. Use according to claim 15, characterized in that the anti-inflammatory product is chosen from the following products: an extract of Glycyrrhiza glabra, glycyrrhetinic acid, an extract of Scutellaria baicalensis, an extract of Aloe vera, bisabolol.

17. Use according to claim 1 of an extract of Griffonia in a cosmetic or dermatological preparation for attenuating the pigmentation of the skin and of superficial body growths, characterized in that said preparation contains at least one agent encouraging flaking.

18. Use according to claim 17, characterized in that the agent encouraging flaking is chosen from the following products: salicylic acid, alpha-hydroxy acids, urea.

19. Use according to claim 1 of an extract of Griffonia in a preparation, characterized in that said extract is incorporated into particles such as liposomes, vesicles, dispersed lamellar phases, capsules, sponges, of size from 1 nm to 100 microns.

20. Use according to claim 19 of an extract of Griffonia in a preparation, characterized in that the size of the particles is from 10 nm to 10 microns.

21. Use according to claim 1 of an extract of Griffonia in a cosmetic or dermatological preparation for attenuating the pigmentation of the skin and of superficial body growths, characterized in that said extract is a powder.

22. Use according to claim 1 of an extract of Griffonia in a cosmetic or dermatological preparation for attenuating the pigmentation of the skin and of superficial body growths, characterized in that said preparation is in the form of an aqueous or hydro-alcohol lotion, an aqueous or hydro-alcohol gel, a micro-emulsion, a liquid or thick emulsion, a loose or compacted powder.

23. Use according to claim 1 of an extract of Griffonia in a cosmetic or dermatological preparation for attenuating the pigmentation of the skin and of superficial body growths, characterized in that said preparation is disposed on a solid support to form a patch.

24. Use according to claim 22 of an extract of Griffonia in a cosmetic or dermatological preparation for attenuating the pigmentation of the skin and of superficial body growths, characterized in that said preparation is in an ingestible form.

25. Use according to claim 24 of an extract of Griffonia in a cosmetic or dermatological preparation for attenuating the pigmentation of the skin and of superficial body growths, characterized in that said preparation is in the form of a capsule or tablet containing from 5 mg to 200 mg of 5-hydroxytryptophan.

26. Use according to claim 25 of an extract of Griffonia in a cosmetic or dermatological preparation for attenuating the pigmentation of the skin and of superficial body growths, characterized in that said preparation is in the form of a capsule or tablet containing from 25 to 75 mg of 5-hydroxytryptophan.
